(19) United States
(12) Patent Application Publication
(10) Pub. No.: US 2025/0281052 A1
(43) Pub. Date: Sep. 11, 2025

(54) PHYSIOLOGICAL MONITORING SYSTEM, STEERING WHEEL AND METHOD FOR PHYSIOLOGICAL MONITORING

(71) Applicant: AUO Corporation, Hsinchu (TW)

(72) Inventor: Yen-Tang HUANG, Hsinchu City (TW)

(21) Appl. No.: 19/068,829

(22) Filed: Mar. 3, 2025

(30) Foreign Application Priority Data
Mar. 8, 2024 (TW) 113108730

(51) Int. Cl. A61B 5/021; A61B 5/00; A61B 5/024; A61B 5/1455; B62D 1/04

(52) U.S. Cl. CPC A61B 5/02141; A61B 5/02427; A61B 5/1455; A61B 5/6893; B62D 1/046

(57) ABSTRACT

A physiological monitoring system for monitoring the physiological state is provided. The physiological monitoring system includes a light source module, a plurality of pixel sensors and a processing unit signally connected to the pixel sensors. The light source module emits the light rays to a recognizable region. A part of the light rays reaches the skin area of the user where the recognizable region touches and transmits to the dermis of the skin area. The pixel sensors are distributed in the recognizable region, and these pixel sensors continuously monitor the ambient light ray detected by the recognizable region and monitor the reflective light ray passing through the dermis of the skin area. Thus, the brightness changing signals are output and converted into touching signals and photoplethysmography signals. The processing unit is used to determine the physiological states of the user based on the touching signals and the photoplethysmography signals.

### RELATED APPLICATIONS

This application claims priority to Taiwan Application Serial Number 113108730, filed Mar. 8, 2024, which is herein incorporated by reference in its entirety.

### BACKGROUND

### Technical field

The present disclosure relates to a physiological monitoring system, a steering wheel using this physiological monitoring and a method for physiological monitoring with the physiological monitoring system.

### Description of Related Art

With the development of smart cars and the improvement of in-car active safety equipment, the model of improving driving safety by detecting the status of drivers has gradually been promoted into the market. The mainstream technology adopts the image recognition to detect the facial expressions of drivers (such as eyes, noses and mouths), the positions of limbs (such as arms) or the body postures to estimate the status of drivers. However, the image recognition is easily interfered by the objects or other passengers in the cars, and thus the misjudgments may occur. For example, when the face of a driver is blocked by the interior decorations in the car, the driver's expression is unable to be determined by the image recognition equipment accurately. In addition, since the process of the image recognition requires a large amount of graphical calculations, a large amount of calculation resources is necessary. As a result, the cost of setting up an image recognition system on the vehicle is high, and the dangers which are caused by the abnormal status of drivers are unable to be predicted.

### SUMMARY

Accordingly, the disclosure is to provide a physiological monitoring system which is able to improve the driving safety.

At least one embodiment of the disclosure provides a steering wheel using the aforementioned physiological monitoring system.

At least one embodiment of the disclosure provides a method of physiological monitoring using the aforementioned physiological monitoring system.

At least one embodiment of the disclosure provides a physiological monitoring system which includes a recognizable region. The physiological monitoring system is used to monitor a physiological state of an user who touches the recognizable region. The physiological monitoring system includes a light source module, a plurality of pixel sensors and a processing unit. The light source module is used to emit a plurality of light rays to the recognizable region, and a part of the plurality of light rays reaches a skin area of the user where the recognizable region touches and transmits to a dermis of the skin area. The plurality of pixel sensors are distributed in the recognizable region, and at least a part of the plurality of pixel sensors is used to continuously monitor an ambient light ray detected by the recognizable region. A plurality of first brightness changing signals are output and converted into a plurality of touching signals, and the plurality of pixel sensors is used to continuously monitor a reflective light ray passing through the dermis of the skin area. A plurality of second brightness changing signals are output and converted into a plurality of photoplethysmography signals. The processing unit signally is connected to the plurality of pixel sensors, and the processing unit is used to determine the physiological state of the user based on the plurality of touching signals and the plurality of photoplethysmography signals.

At least in one embodiment of the disclosure, the light source module includes a plurality of first light-emitting components used to emit the plurality of light rays to the recognizable region with wavelengths between 780 nm and 1500 nm.

At least in one embodiment of the disclosure, the light source module further includes a plurality of second light-emitting components used to emit the plurality of light rays to the recognizable region with wavelengths between 380 nm and 780 nm.

At least in one embodiment of the disclosure, a part of the plurality of second light-emitting components is used to emit the plurality of light rays to the recognizable region with wavelengths between 620 nm and 780 nm, and another part of the plurality of second light-emitting components is used to emit the plurality of light rays to the recognizable region with wavelengths between 495 nm and 570 nm.

At least in one embodiment of the disclosure, the plurality of touching signals includes an area signal of the skin area.

At least in one embodiment of the disclosure, the plurality of touching signals includes a shape signal of the skin area.

At least in one embodiment of the disclosure, the plurality of touching signals includes a distribution signal of the skin area on the recognizable region.

At least in one embodiment of the disclosure, the plurality of photoplethysmography signals includes a heart rate determined signal, an atrial fibrillation determined signal, a blood pressure determined signal and a glycohemoglobin determined signal.

At least one embodiment of the disclosure provides a steering wheel including a grip part and the aforementioned physiological monitoring system. The recognizable region of the physiological monitoring system is distributed on a surface of the grip part.

At least one embodiment of the disclosure provides a method for physiological monitoring. The method includes continuously monitoring an ambient light ray detected by a recognizable region with a plurality of pixel sensors, and a plurality of first brightness changing signals are acquired. The method includes defining a touching region based on the plurality of first brightness changing signals. The method includes converting the plurality of first brightness changing signals into a plurality of touching signals. The method includes emitting a plurality of light rays to a skin area of an user by a light source module. The skin area is located above and overlaps the touching region, and the plurality of light rays transmit to a dermis of the skin area. The method includes continuously monitoring a reflective light ray passing through the dermis of the skin area with the plurality of pixel sensors, and a plurality of second brightness changing signals are acquired. The method includes converting the plurality of second brightness changing signals into a plurality of photoplethysmography signals. The method includes computing a quality verifying standard of physiological signals based on the plurality of photoplethysmography signals. The method includes comparing the plurality of touching signals with the plurality of photoplethysmography signals based on the quality verifying standard of physiological signals to determine a physiological state of the user.

At least in one embodiment of the disclosure, the method further includes emitting an ambient scanning light ray by the light source module when an illumination of the recognizable region is below 50 Lux, and a part of the ambient scanning light ray is reflected by the skin area. The method includes continuously monitoring the part of the ambient scanning light ray by the plurality of pixel sensors.

At least in one embodiment of the disclosure, a wavelength of the ambient scanning light ray is between 780 nm and 1500 nm.

At least in one embodiment of the disclosure, the method further includes filtering out a noise of the plurality of photoplethysmography signals based on a background photoplethysmography signal.

According to the aforementioned embodiments, the pixel sensors of the physiological monitoring system continuously monitor the light rays to acquire the first brightness changing signals and the second brightness changing signals. The first brightness changing signals are converted into the touching signals, and the second brightness changing signals are converted into the photoplethysmography signals. The touching signals may be used to determine the parts and the postures that the user touches on the recognizable region of the physiological monitoring system. The photoplethysmography signals may be converted into the various physiological data (e.g., the heart rate, the atrial fibrillation, the blood pressure and the glycohemoglobin) so as to determine the physiological state of the user. The physiological monitoring system is disposed on the steering wheel. Thus, not only the controlled degree of the vehicle may be determined by the parts and the positions where the user hold the steering wheel, but also the physiological state of the user may be determined by the measurement of various physiological data. Therefore, the risks of vehicle driven state may be accurately determined by the in-car active safety equipment, so that the safety for driving is more completed.

### BRIEF DESCRIPTION OF THE DRAWINGS

To illustrate more clearly the aforementioned and the other features, merits, and embodiments of the present disclosure, the description of the accompanying figures are as follows:

FIG. 1 illustrates a schematic view of a physiological monitoring system in accordance with at least one embodiment of the present disclosure.

FIG. 2 illustrates a locally top view of a physiological monitoring system in accordance with at least one embodiment of the present disclosure.

FIG. 3 illustrates a flow chart of determining the availability of physiological signals based on the photoplethysmography signals in accordance with at least one embodiment of the present disclosure.

FIG. 4 illustrates a schematically top view of a recognizable region in accordance with at least one embodiment of the present disclosure.

FIG. 5A illustrates a data diagram of heart rate determined signals of the photoplethysmography signals at different hand parts in accordance with at least one embodiment of the present disclosure.

FIG. 5B illustrates a data diagram of heart-rate forecast error of the photoplethysmography signals at different hand parts in accordance with at least one embodiment of the present disclosure.

FIG. 6A illustrates a data diagram of atrial fibrillation determined signals of the photoplethysmography signals at different hand parts in accordance with at least one embodiment of the present disclosure.

FIG. 6B illustrates a data diagram of atrial-fibrillation forecast error of the photoplethysmography signals at different hand parts in accordance with at least one embodiment of the present disclosure.

FIG. 7A illustrates a data diagram of blood pressure determined signals of the photoplethysmography signals at different hand parts in accordance with at least one embodiment of the present disclosure.

FIG. 7B illustrates a data diagram of blood pressure deviation of the photoplethysmography signals at different hand parts in accordance with at least one embodiment of the present disclosure.

FIG. 8 illustrates a data diagram of glycohemoglobin determined signals of the photoplethysmography signals at fingers and wrists by the light rays with different wavelengths in accordance with at least one embodiment of the present disclosure.

FIG. 9 illustrates a front view of a steering wheel under the user's controlling situation in accordance with at least one embodiment of the present disclosure.

FIG. 10 illustrates a flow chart of a steering wheel under the controlling situation in accordance with at least one embodiment of the present disclosure.

### DETAILED DESCRIPTION

The following disclosure provides many different embodiments, or examples, for implementing different features of the provided subject matter. Specific examples of components and arrangements are described below to simplify the present disclosure. These are, of course, merely examples and are not intended to be limiting. For example, the formation of a first feature over or on a second feature in the description that follows may include embodiments in which the first and second features are formed in direct contact, and may also include embodiments in which additional features may be formed between the first and second features, such that the first and second features may not be in direct contact. In addition, the present disclosure may repeat reference numerals and/or letters in the various examples. This repetition is for the purpose of simplicity and clarity and does not in itself dictate a relationship between the various embodiments and/or configurations discussed.

Spatially relative terms, such as “beneath,” “below,” “lower,” “above,” “upper” and the like, may be used herein for ease of description to describe one element or feature's relationship to another element(s) or feature(s) as illustrated in the figures. The spatially relative terms are intended to encompass different orientations of the device in use or operation in addition to the orientation depicted in the figures. The apparatus may be otherwise oriented (rotated 90 degrees or at other orientations) and the spatially relative descriptors used herein may likewise be interpreted accordingly.

In the following description, the dimensions (such as lengths, widths and thicknesses) of components (such as layers, films, substrates and regions) in the drawings are enlarged not-to-scale, and the number of components may be reduced in order to clarify the technical features of the disclosure. Therefore, the following illustrations and explanations are not limited to the number of components, the number of components, the dimensions and the shapes of components, and the deviation of size and shape caused by the practical procedures or tolerances are included. For example, a flat surface shown in drawings may have rough and/or non-linear features, while angles shown in drawings may be circular. As a result, the drawings of components shown in the disclosure are mainly for illustration and not intended to accurately depict the real shapes of the components, nor are intended to limit the scope of the claimed content of the disclosure.

Further, when a number or a range of numbers is described with “about,” “approximate,” “substantially,” and the like, the term is intended to encompass numbers that are within a reasonable range considering variations that inherently arise during manufacturing as understood by one of ordinary skill in the art. In addition, the number or range of numbers encompasses a reasonable range including the number described, such as within +/−30%, +/−20%, +/−10% or +/−5% of the number described, based on known manufacturing tolerances associated with manufacturing a feature having a characteristic associated with the number. The words of deviations such as “about,” “approximate,” “substantially,” and the like are chosen in accordance with the optical properties, etching properties, mechanical properties or other properties. The words of deviations used in the optical properties, etching properties, mechanical properties or other properties are not chosen with a single standard.

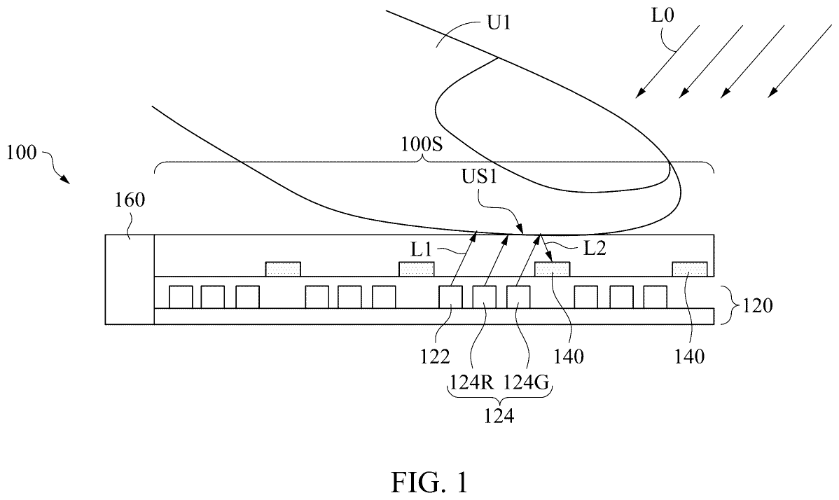

FIG. 1 illustrates a schematic view of a physiological monitoring system in accordance with one embodiment of the present disclosure, and FIG. 1 illustrates a physiological monitoring system 100 in a side view. The physiological monitoring system 100 includes a recognizable region 100S, and the physiological monitoring system 100 is configured to monitor a physiological state of an user U1 touching the recognizable region 100S. The physiological monitoring system 100 includes a light source module 120, a plurality of pixel sensors 140 and a processing unit 160.

The light source module 120 is configured to emit a plurality of light rays L1 to the recognizable region 100S, while a part of the light rays L1 (i.e., some of the light rays L1) reaches a skin area US1 of the user U1 where the recognizable region 100S touches and transmits to a dermis of the skin area US1 (not shown). The skin area US1 is a touching region between the skin (e.g., the skin of fingers or palms) of the user U1 and the recognizable region 100S. In the embodiment, the light source module 120 overlaps the recognizable region 100S and is located below the recognizable region 100S. However, the light source module 120 is not limited to be disposed on the aforementioned position. In other embodiments, the light source module 120 may not overlap the recognizable region 100S.

The light source module 120 includes a plurality of first light-emitting components 122 which are configured to emit the light rays L1 to the recognizable region 100S with wavelengths between 780 nm and 1500 nm (i.e., within the wavelength range of near-infrared light). In other words, a part of the light rays L1 may be the infrared light. In addition, the light source module 120 further includes a plurality of second light-emitting components 124 which are configured to emit the light rays L1 to the recognizable region 100S with wavelengths between 380 nm and 780 nm (i.e., within the wavelength range of the visible light).

Thus, a part of the light rays L1 may be the infrared light, while another part of the light rays L1 may be the visible light. In other words, the wavelengths of the light rays L1 which are emitted to the recognizable region 100S by the light source module 120 may be between the ranges of the infrared light and the visible light. The first light-emitting components 122 and the second light-emitting components 124 may be solid-state light-emitting components, such as organic light-emitting diodes (OLEDs), micro light-emitting diodes (Micro LEDs) or other similar components.

For example, a part of the second light-emitting components 124 (i.e., the light-emitting component 124R in FIG. 1) is configured to emit the light rays L1 to the recognizable region 100S with wavelengths between 620 nm and 780 nm (the wavelength range of the red light), while another part of the second light-emitting components 124 (i.e., the light-emitting component 124G in FIG. 1) is configured to emit the light rays L1 to the recognizable region 100S with wavelengths between 495 nm and 570 nm (the wavelength range of the green light). In other words, the second light-emitting components 124 may emit the red light and the green light to the recognizable region 100S. However, the light-emitting components included in the light source module 120 are not limited to the aforementioned embodiment (i.e., the light-emitting components are not limited to be the first light-emitting components 122 and the second light-emitting components 124). In other embodiments, the light source module 120 may include the light-emitting components within other wavelength ranges, such as blue light-emitting components.

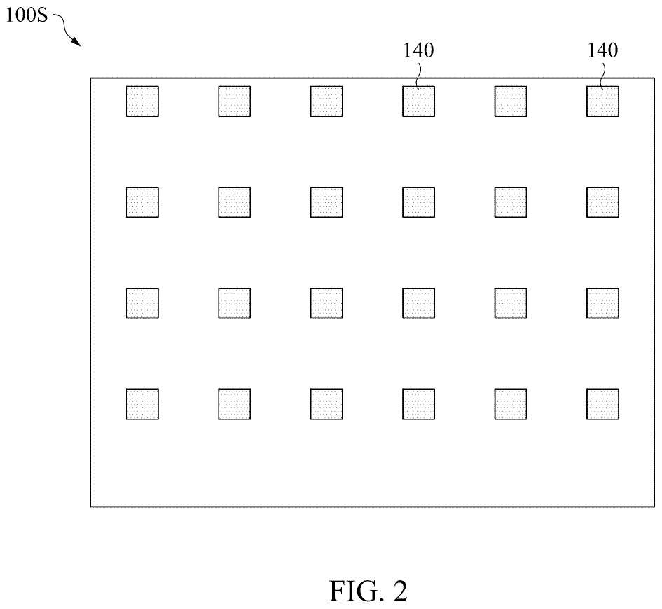

FIG. 2 is a schematically top view of the recognizable region 100S of the physiological monitoring system 100. Referring to FIG. 1 and FIG. 2, the pixel sensors 140 are distributed in the recognizable region 100S, and the pixel sensors 140 are configured to continuously monitor an ambient light ray L0 detected by the recognizable region 100S, so that a plurality of first brightness changing signals are output and converted into a plurality of touching signals. The ambient light rays L0 are the visible light. It is worth mentioning that not all of the pixel sensors 140 are able to monitor the ambient light rays L0 and output the first brightness change signals in the embodiment. In other words, only a part of the pixel sensors 140 (i.e., some of the pixel sensors 140) are able to continuously monitor the ambient light rays L0 detected by the recognizable region 100S, but the disclosure is not limited to the embodiment. In some embodiments, all of the pixel sensors 140 are able to monitor the ambient light rays L0 and output the first brightness change signals.

The pixel sensors 140 may be complementary metal oxide semiconductors (CMOS) active pixel sensors, charge coupled devices (CCD) or similar electronic devices, but the pixel sensors 140 of the disclosure are not limited to aforementioned electronic devices.

When the recognizable region 100S is not touched by the user U1, the ambient light rays L0 may reach each of the pixel sensors 140 on the recognizable region 100S. However, when the recognizable region 100S is touched by the user U1, a part of the pixel sensors 140 on the recognizable region 100S may be shielded by the user U1, so that the ambient light rays L0 may not reach the recognizable region 100S. By continuously monitoring, the pixel sensors 140 may monitor the brightness change between the aforementioned conditions so as to produce the first brightness change signals. Since the first brightness change signals may be changed by the area of the recognizable region 100S that is touched by the user U1, and thus the touching information between the user U1 and the recognizable region 100S may be acquired by converting the first brightness change signals into the touching signals.

Specifically, in the embodiment, the touching signals include the area and the shape of a skin area US1 that the recognizable region 100S is touched by the user U1, and the touching signals further include the distribution of the skin area US1 on the recognizable region 100S. The processing unit 160 may determine the way that the recognizable region 100S is touched by the user U1 based on the area, the shape and the distribution of the skin area US1 on the recognizable region 100S. For example, the result that the recognizable region 100S is touched by the fingers of the user U1 instead of the palms of the user U1 may be determined due to the difference between the shapes of the fingers and the palms.

The processing unit 160 is signally connected to the pixel sensors 140. It is worth mentioning that the position where the processing unit 160 is disposed in FIG. 1 is schematic. In various embodiments of the disclosure, the position where the processing unit 160 may be disposed depends on the requirement of application.

In addition, at least a part of the pixel sensors 140 are configured to continuously monitor a reflective light ray L2 which passes through the dermis of the skin area US1, so that a plurality of second brightness changing signals are output and converted into a plurality of photoplethysmography signals. The wavelength of the reflective light ray L2 is between the range of the visible light (i.e., the wavelengths between 380nm and 780 nm) and the infrared light (i.e., the wavelengths between 780 nm and 1500 nm). In the embodiment, the photoplethysmography signals include a heart rate (HR) determined signal, an atrial fibrillation (AFib) determined signal, a blood pressure (BP) determined signal and a glycohemoglobin (HbAlc) determined signal. After the aforementioned touching signals and the photoplethysmography signals are acquired, the processing unit 160 determines the physiological state of the user based on the touching signals and the photoplethysmography signals.

It is worth mentioning, the photoplethysmography (PPG) is a non-invasive biological detection technology that detects the changes of blood volume in living tissues by the conversion of photoelectric signals. The technology utilizes light beams in a specific wavelength range to illuminate the skin surface. When the light beams pass through the skin tissues and then be reflected to the photosensitive components, the intensity of the light beams will be weakened. Since the blood is flowing in blood vessels, the light absorption rate of the blood will be changed due to the change of blood flow. Furthermore, the vasoconstriction and vasodilation during the heartbeat cause the changes of the blood flow, thereby affecting the reflectivity of the skin surface to the light beams. As a result, the intensity of the light beams which is detected by the photosensitive components is changed. When the change of the intensity of the light beams is converted into the electronic signals (i.e., the photoplethysmography signals), and the electronic signals are analyzed, the physiological information related to the blood and heartbeat may be acquired.

The photoplethysmography signals are spectral signals. Specifically, in the embodiment, the heart rate determined signal is determined by the power spectral density (PSD) of the photoplethysmography signals. The atrial fibrillation (AFib) determined signal is determined by the product of the intensity of the photoplethysmography signals (i.e., the intensity of AC/DC signals) and the power spectral density. The blood pressure determined signal is determined by the quality check rate (QC rate) of the photoplethysmography signals. In other words, the blood pressure determined signal depends on the result that whether the waveform distribution of the photoplethysmography signals conforms to a good pulse contour characteristic. The glycohemoglobin determined signal is determined by the quality of the photoplethysmography signals.

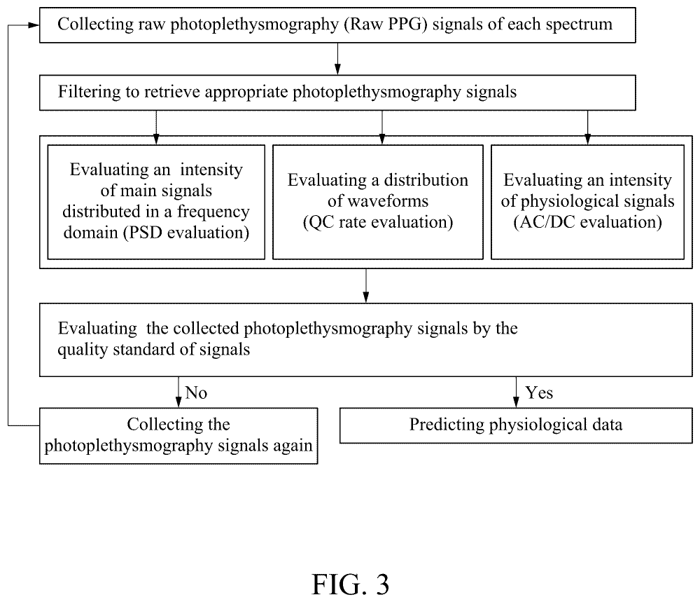

Referring to FIG. 3 illustrating a flow chart of determining the availability of physiological signals based on the photoplethysmography signals, the raw photoplethysmography signals may be filtered so as to retrieve the appropriate data for determining physiological signals after the raw photoplethysmography signals are collected. These photoplethysmography signals include the power spectral density, the intensity of AC/DC signals and the quality check rate. Thus, the quality of those collected photoplethysmography signals may be evaluated by each quality standard of signals. If the quality does not meet the quality standard, the photoplethysmography signals may be collected again. If the quality meets the quality standard, the various physiological data may be predicted.

A quality verifying standard of physiological signals based on the photoplethysmography signals is computed in at least one embedment of the disclosure so as to infer the usability of physiological signals which are received subsequently for determining the physiological state. Further, the accuracy of determining the physiological state may be improved.

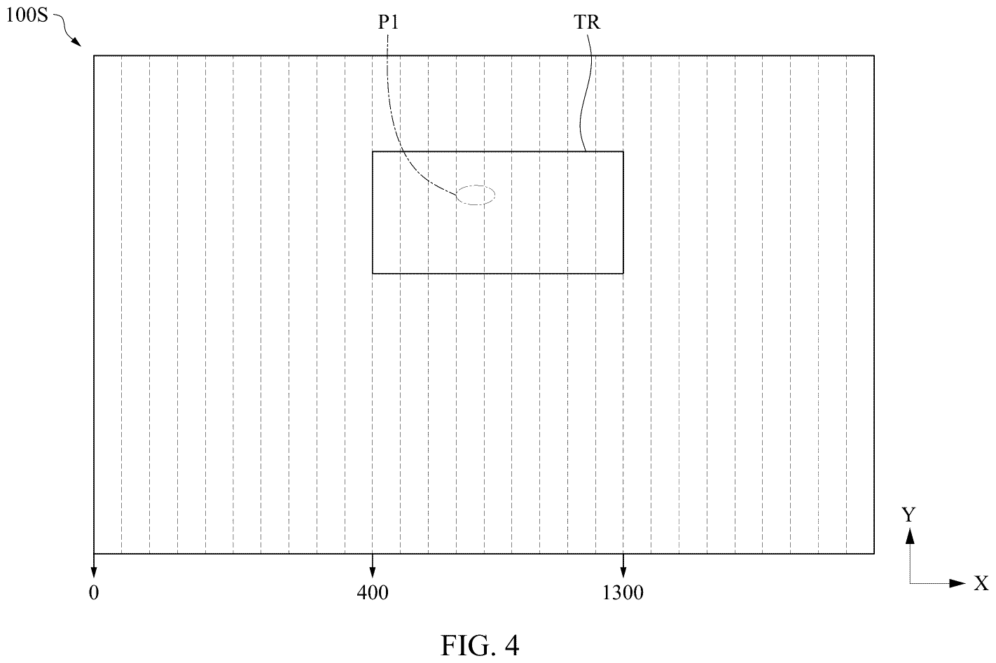

Referring to FIG. 4, the following embodiment is an example of a photoplethysmography sensor with the red light, and the detection of determining standard for aforementioned physiological signals is performed on one of the testing region TR of the recognizable region 100S so as to acquire the determining standard of various physiological data. For example, the (red light) pixel sensors 140 are distributed in the recognizable region 100S and arranged in a 40×40 matrix (i.e., the matrix with 40 rows and with 40 columns). A part of the pixel sensors 140 are distributed in the testing region TR. The different parts of hands (such as a finger, an upper edge of the palm, an outer edge of the palm, a midpoint of the wrist and a radial artery of the wrist) are pressed on the pressing point P1 respectively, and the range of interest (ROI) along the X-axis of the center of the testing region TR is within the pixel sensors 140 ranging from numbers 400 to 1300 along the X-axis. After the detection by the pixel sensors 140, multiple sets (i.e., different parts of hands) of data of the photoplethysmography signals at different positions within the range of interest can be acquired.

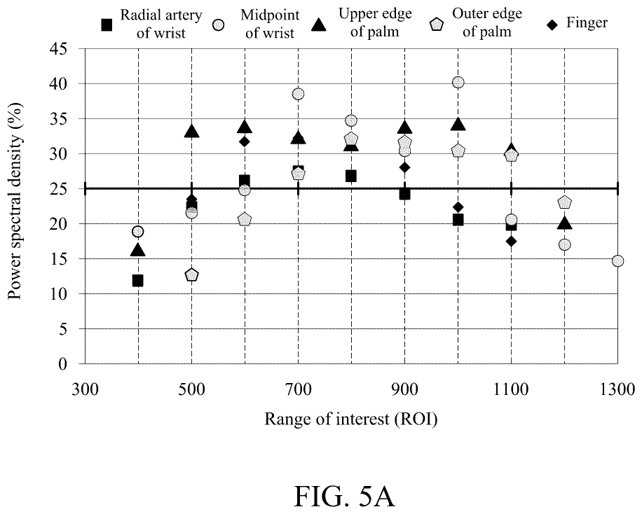

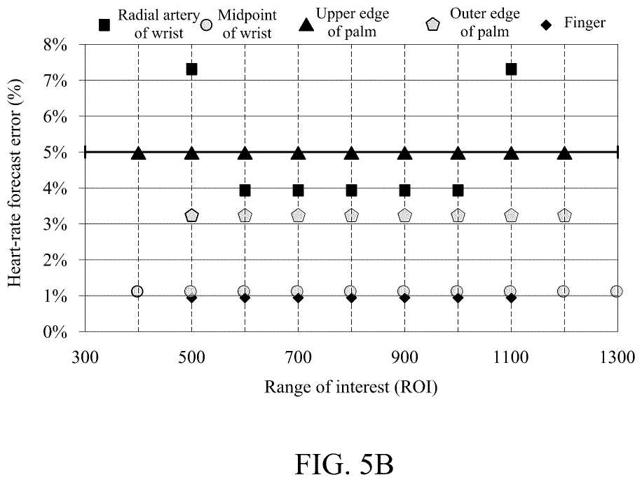

As far as the heart rate determined signal is concerned, the power spectrum density should be over 25% so as to be an accurate criterion of the heart rate without misjudgments. Referring to FIG. 5A and FIG. 5B, the Y-axis of FIG. 5A represents the power spectrum density in percentage, while the Y-axis of FIG. 5B represents the heart-rate forecast error in percentage. According to FIG. 5A, although all tested parts (of hands) have multiple data over 25% within the range of interest from numbers 400 to 1300, only three data of the radial artery of the wrist which are closest to the pressing point P1 (the range from numbers 650 to 850 approximately) are 2% to 3% more than 25%. In other words, the heart rate signal received from the radial artery of the wrist within the range of interest is weak. Thus, a larger error may be acquired when this part (of hands) is used as the criterion for the heart rate signal. As shown in FIG. 5B, only the heart rate determined signal of the radial artery of the wrist performs obvious errors (i.e., the heart-rate forecast error is over 5%), while the heart rate determined signals of the finger, the upper edge of the palm, the outer edge of the palm, the midpoint of the wrist perform barely errors.

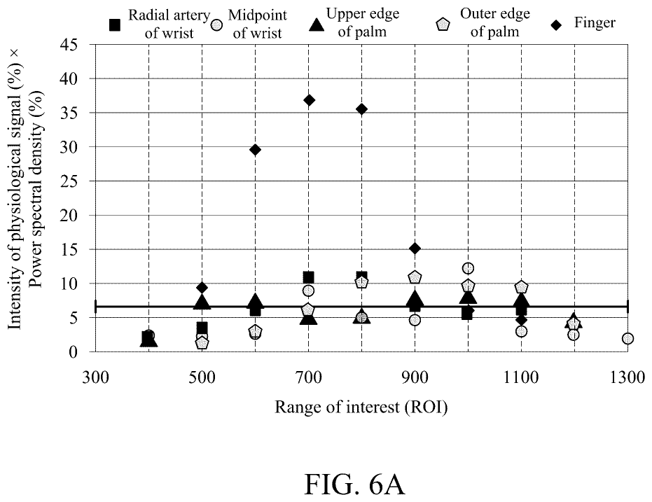

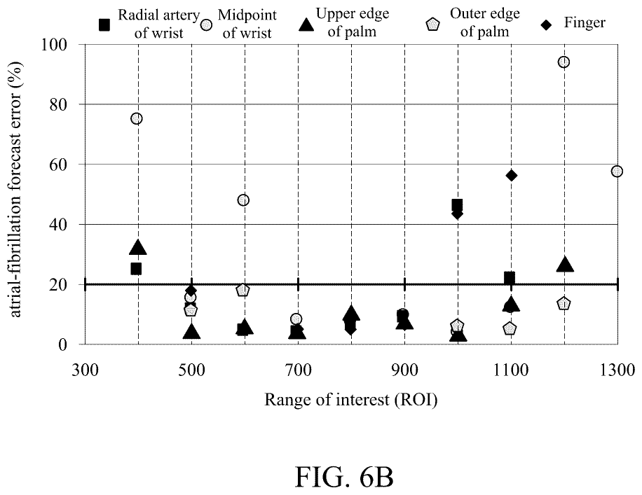

As far as the atrial fibrillation determined signal is concerned, the product of the intensity of physiological signal and the power spectral density should be over 6.5% % so as to be a criterion of the atrial fibrillation. Referring to FIG. 6A and FIG. 6B, the Y-axis of FIG. 6A represents the product of the intensity of physiological signal in percentage and the power spectrum density in percentage, while the Y-axis of FIG. 6B represents the atrial-fibrillation forecast error in percentage. According to FIG. 6A, although all tested parts (of hands) have multiple data over 6.5‰ within the range of interest from numbers 400 to 1300, only the finger and the outer edge of the palm have four data over 6.5‰. The quantity of data over 6.5‰ of other parts is less than two. In other words, the atrial fibrillation signals received from the finger and the outer edge of the palm within the range of interest is strong. Thus, a smaller error may be acquired when these parts (of hands) are used as the criterion for the atrial fibrillation signal. As shown in FIG. 6B, the atrial fibrillation determined signals of the upper edge of the palm, the midpoint of the wrist and the radial artery of the wrist perform obvious errors (i.e., the atrial-fibrillation forecast error is over 20%), while only the atrial fibrillation determined signals at the farthest ends from the pressing point P1 of the finger and the outer edge of the palm perform obvious errors. The errors of the other positions (of hands) are not obvious.

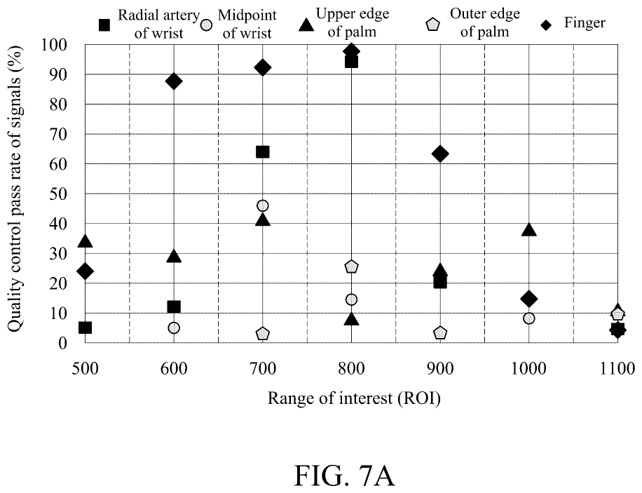

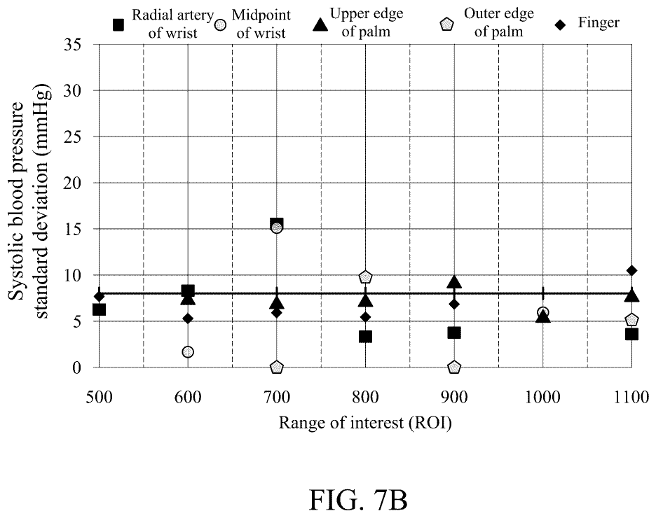

As far as the blood pressure determined signal is concerned, the quality control pass rate of signals should be over 20% so as to be a criterion of the blood pressure (the quality control pass rate of signals is larger-the-better). Referring to FIG. 7A and FIG. 7B, the Y-axis of FIG. 7A represents the quality control pass rate of signals in percentage, while the Y-axis of FIG. 7B represents the systolic blood pressure standard deviation (SBP STD). According to FIG. 7A, the finger, the radial artery of the wrist and the upper edge of the palm have at least two data over the criterion (i.e., 20%) within the range of interest from numbers 400 to 1300. All of the data of the other parts (of hands) are not over the criterion. In other words, the quality of the photoplethysmography signals received from the finger, the radial artery of the wrist and the upper edge of the palm is better within the entire range of interest.

It is worth mentioning that the systolic blood pressure standard deviation is not affected by the quality control pass rate of signals in the embodiment. To take the systolic blood pressure as an example, as shown in FIG. 7B, most of the systolic blood pressure standard deviations of the finger, the radial artery of the wrist and the upper edge of the palm is less than or equal to 8 mmHg. Among aforementioned three parts, each has one data over 8 mmHg, and the distributed range of data points is concentrated. The error may be corrected by subsequent artificial intelligences, so that the blood pressure error is within the acceptable range. However, since the distributed range of data points of the midpoint of the wrist and the outer edge of the palm is divergent, the effective correction is unable to be achieved. Two data of the midpoint of the wrist are over 8 mmHg, so that these data are determined to have larger errors. Furthermore, although two data of the outer edge of the palm are close to 0 mmHg, the data of the outer edge of the palm is determined to have a larger error since one data of the outer edge of the palm is over 8 mmHg.

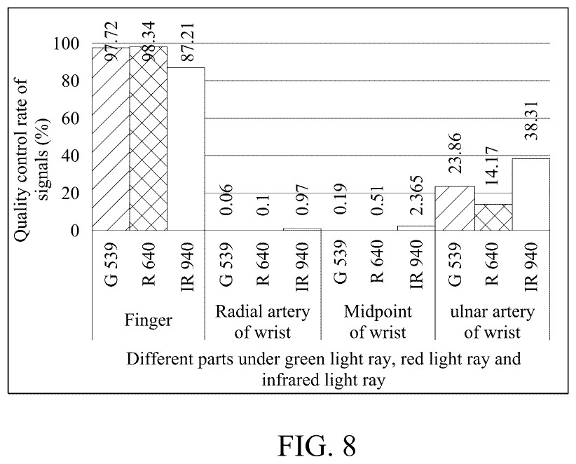

As far as the glycohemoglobin determined signal, the light rays in different wavelength ranges may be used as the light sources for determining the photoplethysmography signals, such as the green light rays, the red light rays, and the infrared light rays. The data of glycohemoglobin can be determined based on the quality of the photoplethysmography signals generated by the light rays in different wavelength ranges. Referring to FIG. 8, the Y-axis of FIG. 8 represents the quality control rate of signals in percentage, where the quality control rate of signals is higher, the quality of signals is better. As shown in the bar chart of FIG. 8, the finger and the wrist are detected by the light rays with different wavelengths. For the detection of the finger, the quality control rate of signals produced by the green light rays (referring to the bar with slash lines in FIG. 8) is 97.72%. The quality control rate of signals produced by the red light rays (referring to the bar with grids in FIG. 8) is 98.34%. The quality control rate of signals produced by the infrared light rays (referring to the hollow bar in FIG. 8) is 87.21%, which is the lowest quality control rate of signals.

In addition, the ulnar artery of the wrist is detected by the light rays with different wavelengths. The quality control rate of signals produced by the infrared light rays is 38.31%. The quality control rate of signals produced by the green light rays is 23.86%, while the quality control rate of signals produced by the red light rays is 14.17%, which is the lowest quality control rate of signals. It is worth mentioning that the quality control rates of signals of the radial artery of the wrist and the midpoint of the wrist detected by the light rays with different wavelengths are less than 2.37%. Thus, it is difficult for the data of glycohemoglobin to be determined.

According to the determination of the aforementioned physiological data, the following conclusion can be drawn. As determining the physiological state of the user U1 by the physiological monitoring system 100, the parts of the user U1 touching the recognizable region 100S may be discern by the touching signals. Thus, the physiological data may be further determined based on the photoplethysmography signals of different touching parts.

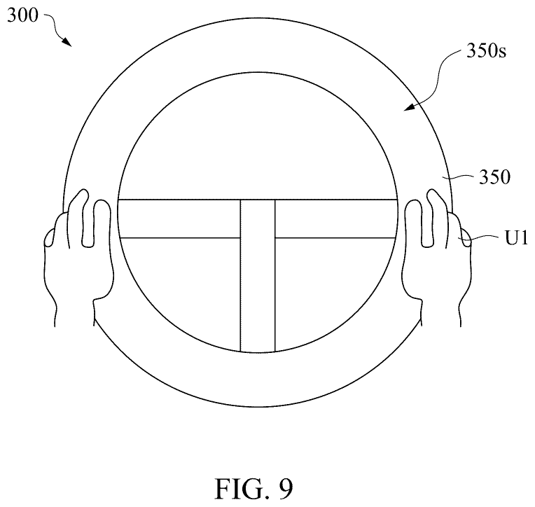

In the embodiment, the physiological monitoring system 100 is disposed on a steering wheel 300. FIG. 9 illustrates the front view of the steering wheel 300 under a controlling condition of the user U1. The steering wheel 300 includes a grip part 350 and the physiological monitoring system 100 (not shown in FIG. 9), while the recognizable region 100S of the physiological monitoring system 100 is distributed on a surface 350s of the grip part 350. When the user U1 (i.e., the driver of a vehicle) controls the steering wheel 300, the physiological state of the user U1 may be monitored by the physiological monitoring system 100 so as to determine whether the vehicle driven by the user U1 is in a safe condition.

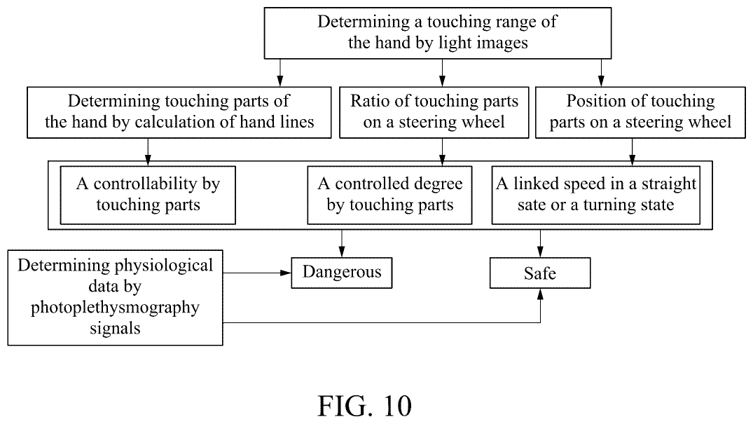

Referring to the flow chart of the steering wheel under the controlling condition in FIG. 10, firstly, the touching range of the hand is determined by the light images. The touching parts of the hand (e.g., the palm or the finger) are recognized based on the calculation of the hand lines after the touching range of the hand is determined. After acquiring the aforementioned information of the touch, the controllability, the controlled degree of the vehicle and the linked speed in a straight state or a turning state by the touching parts may be determined by the aforementioned touching signals. Based on the physiological data of the user determined by the photoplethysmography signals and further based on the driven state determined by the aforementioned touching signals, the safety of the vehicle during driving is confirmed.

Specifically, referring to FIG. 1, FIG. 9 and FIG. 10, the touching state of the user U1 with the recognizable region 100S may be determined by the aforementioned information, such as the area, the shape, and the distribution on the recognizable region 100S of the skin area US1 of the user U1 (i.e., the area where the user U1 touches the recognizable region 100S). For example, it may be determined by the area or the shape of the skin area US1 that the user U1 touches the recognizable region 100S with the finger or the palm, so that the controlled degree of the vehicle by the user U1 may be determined. In addition, since the driver holds specific positions of the steering wheel 300 in individual ways when the driver controlling the vehicle to turn or go straight, the driving state of the vehicle may be determined by the distribution of the skin area US1 on the recognizable region 100S.

In at least one embodiment of the disclosure, the physiological state monitoring method of the physiological monitoring system 100 is as follows. Referring to FIG.1, firstly, the ambient light ray L0 detected by the recognizable region 100S is continuously monitored with the pixel sensors 140, so that the plurality of first brightness changing signals are acquired. Under the strong sunlight, the touching region may be defined based on the first brightness changing signals (e.g., the area with low signals is defined as a shielding area, while the area with high signals is defined as a light-receiving area). The first brightness changing signals are converted into the plurality of touching signals. Specifically, a part of the recognizable region 100S is shielded by the user U1 after the user U1 touches the recognizable region 100S, so that the brightness of the ambient light ray L0 received by the pixel sensors 140 changes. As a result, the touching region between the user U1 and the recognizable region 100S may be determined based on the change of the brightness.

It is worth mentioning that the brightness change which is caused by the shield of the user U1 from the ambient light ray L0 may not be detected in all situations by the pixel sensors 140. For example, the pixel sensors 140 are unable to detect sufficient first brightness change signals to define the touching region of the user U1 on the recognizable region 100S when the brightness of the ambient light ray L0 is weak (e.g., at night or under the dark sky).

As a result, in the embodiment, the method for physiological monitoring further includes emitting an ambient scanning light ray (not shown) by the light source module 120 when the illumination of the recognizable region 100S is less than or equal to 50 Lux. A part of the ambient scanning light ray may be reflected by the skin area US1, while the part of the ambient scanning light ray may be continuously monitored by the pixel sensors 140. It is worth mentioning that the wavelength of the ambient scanning light ray is between 780 nm and 1500 nm in some embodiments so as to prevent the driver from sight interference. In other words, the ambient scanning light ray may be but not limited to the infrared light.

Next, the light source module 120 emits the light rays L1 to the skin area US1 of the user U1. It is worth mentioning that the skin area US1 is located above and overlaps the touching region, and the light rays L1 transmit to the dermis of the skin area US1. In addition, the reflective light ray L2 which passes through the dermis of the skin area US1 is continuously monitored by the pixel sensors 140, so that the second brightness changing signals are acquired. The second brightness changing signals are converted into the photoplethysmography signals, and the physiological state of the user U1 is determined based on the touching signals and the photoplethysmography signals.

It is worth mentioning that the method for physiological monitoring further includes filtering out a noise of the photoplethysmography signals based on a background photoplethysmography signal in some embodiments. In the embodiment, the relative displacement of the testing region TR to the fixed pressing point P1 is used to simulate the situation of signals, and those signals are produced by the unconscious touches of the subject on the recognizable region 100S. Furthermore, the feasibility of each test of the physiological signals is determined. Firstly, the quality of the photoplethysmography signals is evaluated, and then a two-stage calculation method for the prediction of the physiological state is performed. Thus, a better quality of signals may be acquired, and thereby improving the accuracy of the determination of the physiological signals.

In conclusion, the pixel sensors of the physiological monitoring system continuously monitor the light rays to acquire the brightness changing signals (i.e., the first brightness changing signals and the second brightness changing signals), and the brightness changing signals are converted into the touching signals and the photoplethysmography signals. The touching signals may be used to determine the parts and the postures that the user touches on the recognizable region of the physiological monitoring system. The photoplethysmography signals may be converted into the various physiological data (e.g., the heart rate, the atrial fibrillation, the blood pressure and the glycohemoglobin) so as to determine the physiological state of the user.

Moreover, the physiological monitoring system is disposed on the steering wheel. Thus, not only the controlled degree of the vehicle may be determined by the parts and the positions where the user hold the steering wheel, but also the physiological state of the user may be determined by the measurement of various physiological data. Therefore, the risks of vehicle driven state may be accurately determined by the in-car active safety equipment, so that the safety for driving is more completed.

Although the embodiments of the present disclosure have been disclosed as above in the embodiments, they are not intended to limit the embodiments of the present disclosure. Any person having ordinary skill in the art can make various changes and modifications without departing from the spirit and the scope of the embodiments of the present disclosure. Therefore, the protection scope of the embodiments of the present disclosure should be determined according to the scope of the appended claims.

What is claimed is:

1. A physiological monitoring system comprising a recognizable region, wherein the physiological monitoring system is configured to monitor a physiological state of an user touching the recognizable region, and the physiological monitoring system comprises:
a light source module configured to emit a plurality of light rays to the recognizable region, wherein a part of the plurality of light rays reaches a skin area of the user where the recognizable region touches and transmits to a dermis of the skin area;
a plurality of pixel sensors distributed in the recognizable region, wherein at least a part of the plurality of pixel sensors is configured to continuously monitor an ambient light ray detected by the recognizable region, and a plurality of first brightness changing signals are output and converted into a plurality of touching signals, wherein the plurality of pixel sensors is configured to continuously monitor a reflective light ray passing through the dermis of the skin area, and a plurality of second brightness changing signals are output and converted into a plurality of photoplethysmography signals; and
a processing unit signally connected to the plurality of pixel sensors, and the processing unit is configured to determine the physiological state of the user based on the plurality of touching signals and the plurality of photoplethysmography signals.

2. The physiological monitoring system of claim 1, wherein the light source module comprises:
a plurality of first light-emitting components configured to emit the plurality of light rays to the recognizable region with wavelengths between 780 nm and 1500 nm.

3. The physiological monitoring system of claim 2, wherein the light source module further comprises:
a plurality of second light-emitting components configured to emit the plurality of light rays to the recognizable region with wavelengths between 380 nm and 780 nm.

4. The physiological monitoring system of claim 3, wherein a part of the plurality of second light-emitting components is configured to emit the plurality of light rays to the recognizable region with wavelengths between 620 nm and 780 nm, and another part of the plurality of second light-emitting components is configured to emit the plurality of light rays to the recognizable region with wavelengths between 495 nm and 570 nm.

5. The physiological monitoring system of claim 1, wherein the plurality of touching signals comprises an area signal of the skin area.

6. The physiological monitoring system of claim 1, wherein the plurality of touching signals comprises a shape signal of the skin area.

7. The physiological monitoring system of claim 1, wherein the plurality of touching signals comprises a distribution signal of the skin area on the recognizable region.

8. The physiological monitoring system of claim 1, wherein the plurality of photoplethysmography signals comprises a heart rate determined signal, an atrial fibrillation determined signal, a blood pressure determined signal and a glycohemoglobin determined signal.

9. A steering wheel comprising:
a grip part; and
the physiological monitoring system of claim 1, wherein the recognizable region of the physiological monitoring system is distributed on a surface of the grip part.

10. A method for physiological monitoring comprising:
continuously monitoring an ambient light ray detected by a recognizable region with a plurality of pixel sensors, and a plurality of first brightness changing signals are acquired;
defining a touching region based on the plurality of first brightness changing signals;
converting the plurality of first brightness changing signals into a plurality of touching signals;
emitting a plurality of light rays to a skin area of an user by a light source module, wherein the skin area is located above and overlaps the touching region, and the plurality of light rays transmit to a dermis of the skin area;
continuously monitoring a reflective light ray passing through the dermis of the skin area with the plurality of pixel sensors, and a plurality of second brightness changing signals are acquired;
converting the plurality of second brightness changing signals into a plurality of photoplethysmography signals;
computing a quality verifying standard of physiological signals based on the plurality of photoplethysmography signals; and
comparing the plurality of touching signals with the plurality of photoplethysmography signals based on the quality verifying standard of physiological signals to determine a physiological state of the user.

11. The method of claim 10, further comprising:
emitting an ambient scanning light ray by the light source module when an illumination of the recognizable region is below 50 Lux, wherein a part of the ambient scanning light ray is reflected by the skin area; and
continuously monitoring the part of the ambient scanning light ray by the plurality of pixel sensors.

12. The method of claim 11, wherein a wavelength of the ambient scanning light ray is between 780 nm and 1500 nm.

13. The method of claim 10, further comprising:
filtering out a noise of the plurality of photoplethysmography signals based on a background photoplethysmography signal.

14. The method of claim 10, wherein the plurality of photoplethysmography signals comprises a heart rate determined signal, an atrial fibrillation determined signal, a blood pressure determined signal and a glycohemoglobin determined signal.

15. The method of claim 10, wherein the plurality of touching signals comprises an area signal of the skin area, a shape signal of the skin area and a distribution signal of the skin area on the recognizable region.
